SEQUENCE LISTING (7 sequences)

SEQ ID NO: 1 - protein, 414 aa, synthetic construct
  source 1..414: note = Synthetic phytase (WO 2012/143862; as phytase PhV-99)
SETEPSGYQLEKVVILSRHGVRAPTKMTQTMRDVTPHQWPEWPVKLGYITPRGEHLISLMGGFYRERFQQQGLLPNDTCPTPDAVYVWTDVNQRTRKTGEAFLAGLAPQCDLAIHHQQNITQADPLFHPVKAGICSMNKSQTYAAVEKQAGGPIETLNQRYQAELALMSSVLDFPKSPYCQQHNIGKLCDFSQAMPSRLNISDDGNEVQLEGAVGLSSTLAEIFLLEYAQGMPVVAWGNIHNESQWKSLLNLHNAHFNLMHRTPYIAKHQGTPLLQAISNALNPNATESKLPDISPDNKILFIAGHDTNIANIGGMLGMNWTLPGQPDNTPPGGGLVFELWQNPDNHQQYVAVKMIYQTMDQLRNSEKLDLKSNPAGIVPIEIEGCENIGTDKLCQLDTFQKRVAQVIEPACQI

SEQ ID NO: 2 - DNA, 10314 bp, synthetic construct
  source 1..10314: mol_type = other DNA; plasmid = Plasmid MT2703
ggccgcatcgaattcctgcagcccgggggatccgcccgggctaggcgccgagcggccgccaccgcggtgagctctcgatcggtcgtgctatgctagttattgctcagaatcagctgcattaatgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtatgcatctagtgcttggattctcaccaataaaaaacgcccggcggcaaccgagcgttctgaacaaatccagatggagttctgagttaattaatggatctatcaacaggagtccaagcaagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccgcccacagtcgatgaatccagaaaagcggccattttccaccatgatattcggcaagcaggcatccccatgtgtcacgacgagatcctcgccgtcgggcatacgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatctgatcttgatcccctgcgccatcagattcttggcggcaagaaagccatccagtttactttgcagggcttcccaaccttaccagagggcgccccagctggcaattccgacgtttagctgcggccgctacgtagtttaaacgtgatgccgcggtcctcgttggtctgggctcttctcatttcccggtttgcaccccccgttgcacctgctgatcgcccgccaacgccgatgaggttgcgcccagaccgacaatcaccgcggctgcattcccaagtatattgaagatggcaccaggtacccggttttgcgtcccagtcgtttggtgccaaatttgggagtttttgagcctcaagatctggggaaatcgacctcaacttccatacaagttaaagtcgcacacacggcgagttccacgaagagacacatttttttctgaaggcctctctccccgcacatcagaaaccaccaaataccaagactgcagaagccggggtaagtgggccaccgggactacactaaaatgcggggagaagcgagatccgttgcgaagggaagggatggggtgtgctgcggctttctccgctctcgtgcgccttttgcttgaatctagtgtacaccagggtaggctccgaaggagtatctacggcagcgctgttcgtgctgcgttgagagtcagggcggagacgagcaggcgacaggagcctcgcaccggcacttcggatcgcatttgcgcggagcgtcaaatacgctcttctgcggtcatcagagagcatcgtgaaccaaggttcttccgcagggcggcctgggcttcgcagagtcgcactcggcggacgccttccgtgtcacccctgataacctggctgccgcgcccagactcctccaatgaggtgtgtggttgccctcgccgacccttcagcaaccttaatcgcttccatcgcacggctccacgtcctcgaacgatgccctcagtccgtgcccggccgtggcaaccataacgtgacatcgccgcccagcctactagccgctatcgaccggttaggcttgtcaccgcagcgcccattctccatcgggcctctactctgatccacctcacccaccgcaagcactagcgagcctcaccagagtgcaagcgacacgacccgcttggcccttcgtccttgactatctcccagacctcttgccatcttgccgacgccgcccccttttttttctcctccccctgccggcaggtcggtggccccagtcccgagatggcattgctccgttgtccatgacgacccatcattcgatggctgactggcacactcgtcttgtttgagcatcgacggcccgcggcccgtctcccacggtacggaacctcgttgtacagtacctctcgtaatgatacccaacaccggggccgagcgctgggagggcggcgttcccgagaagccgggaaggcggctggccggctgacctttgtgacttggcgatggatgcggccatggagaatgtccgtccgaagcgacgcgacaattagcctggctaccatcgatataaattgggtgattcccagctcttgatgggcgtgtcttctgctcgaccctgacagagacgtatcacagaggactgggggtggttcaaaataccagttagtgagaaagacctctcgatgtgaaagggtaggtgctccctccaacttaacacaaaaacgacacactaccaaacctctagaggtggacaaagcaaccccgcgtactccgttccattcgtagcacaggacgccagagaaagcaaaccacaggtcaggaaacacacaaccccgggtttctaggatcgtcggccgtcgtttcacgtatcggctacggaggttagtaagaatcccccgggtgggggggttcctcttgttgaaatgggtgacatttctatgttctgcgcatgctttcagcccagcagcgagcggctcgggtatgctaggcaatcttggagctttgatgtacctaggcgcgtgtgataagaacacaacaaggtccacttcccgtacctaccgcctgtggatcccggttcgaggttggacctgtgcagtaaagcgcacagctgaggacgatttgaggacgattgaggacggacatacctgatgtaggcaacgaaaagcttaagccggcttctacggggagcttctccccatcaccaaagatcggaccacctaggcggctaccgggcatgagatacgagcagggggaagcggcgagtgacacgtcgtccgcccatgggacggctgaaggggtaatggcagcaccttgacgtcccttctcgcggagaggcgggaaccattgctctgggaacggacagctgccgggtgcgccgatactaacgaggttccctggaatatgatggtggaaccgcgctcgcgaggaagcttggccggccaacccctgttcccccgaagacctgtcaccgtcgggaactgccgagctcgaaagaccacagtctccgaattgatcaactcctacttatcccatctatgagaggagaacatcgaacgattctgacccgggaactctggagatgcccatggtgccttcctttagatctggccatgttccgcatctatctacatcagttcagtctttgccctgttggtcttgctagcggcgatgctatgaaaatctatcaattaatggtgtcctttttcgatgcgagtacactactcatttcgtttccgaatcgccatctcttcgaccattgctgaactaattcaatcttaggtagtctgctgttccttgttttccatttccttgtcttaagtaaaaccaactggcacacctcgaaacacgcttgacggatggacagtagaattgaccgtgtacgtacatgtaccttgacgtcctccgaggttcgacatcagggttcgtcatagggagtgaaacacccgccatgattccgtagccgcgcgcgaagatacgaagcagatatttcacggacatggcggagatacttgtttcccgtactaaggtagtcatgtcggagacatctgaacgacagagctggccaagagaaccgaccagttgccccaggacgatctagacaaaaaaaagagagatgagtgggccacttttgccacaacatcgacggccctgcgaccgcccccaggcaaacaaacaaaccgccgaacaataatacttttgtcattttaggaggagcgttgtatggataaaaacaacatctcgttgctgcagaatgtggacttcaaacttgcagaaaatgggaggcggatttgcatgatcggagggtagttgactcacgccgcaggctgcaaatccgtcctccattattccatgaacaacttcgtaaggttgggctgagcgccaatgcctaacggaccgggggccacagcgcaacgtcccacttaaaggccagcgtgacatgccagttccataccaagtagtggcaccagaggcggccaatgctcagtaagggcagggagggaggctcaaacgattggcaaaaagaggggcttgccagttcagttccctgtgcgagcgcgagaggggcagtttcaaatctggaggggtgtgttgcgctggtctgaagagaaagagaagactgtacttaataattgttcaaagagtccatcatcgcgttgcggactcctctagctgtatttagagccctatcattacttgtcgggtgcgaatcaaaataccgggatgcagccctctggcgatttgcatgcggttgtggaggaagtgaagcctgaatcgcggggctgggcggcaaagcacgacgtgaaattcctggcgaaattcgagggcttgccccaccgtggttgaagtttttgtgctgcgtaaccccaccaacccgccttgcccctcccgcctgcccataaaaacttcgacccctcctcaaatcttcttcgattcttcctcttcacttccttcgtcggcatacctgattcaagcaatcacctgccactttcaagtgcgtataccatcatcgatacactggttcttgacaagtacatcgtctctaactttcctttttgcagttttcattaagcgcaagtcgccagtttcgttcttcagaacacaaataccgtcaccatggtgactccctctctgaagaaggcagctctggctgccctgtccctcttcccgctcctctccctggctagcgaaaccgaaccttccggatatcagctggagaaggtggtcattctctcgcgtcacggtgtccgagcccccaccaagatgacacagacgatgcgcgatgtcactcctcatcagtggcctgagtggcccgtgaagctcggctacatcactcctcgtggagaacacctcatcagcctgatgggcggtttctatagggaacggttccagcagcagggattgcttcccaacgacacctgtccgacccccgacgccgtctacgtgtggaccgacgttaaccagcgtacccgcaagactggagaggctttcctcgccggtcttgcgcctcagtgtgatctggccatccaccaccagcagaacatcacgcaggctgacccgctgtttcacccggtcaaggccggtatctgttcgatgaacaagtctcagacctatgcggctgtcgagaagcaggctggcggccctattgagacgctaaaccagcgctaccaggccgaactggcattgatgtcctctgtgttggatttccccaagtccccatattgccagcagcataacatcggcaaactgtgcgacttttcacaggctatgcctagccgcctcaacatctccgatgacgggaatgaggtgcaactcgaaggcgccgtcggtctttcctccacgctcgccgagatcttcctactggaatacgctcagggtatgcctgtggtcgcctggggcaacattcacaacgagagccagtggaagagcctccttaacttgcacaacgcccatttcaacctgatgcacagaacgccctacattgccaagcaccagggaacccctttacttcaggctatcagcaacgctctcaacccaaatgcaactgagtcgaagctccccgatatctctcccgacaacaagatccttttcattgccggccacgacaccaacatcgcaaacatcggaggcatgttgggtatgaactggactctcccgggccagccagacaatactccgcccggcggtggactggttttcgaactctggcagaacccggataaccatcagcagtacgttgcggtgaagatgatctaccagaccatggaccagctgcgcaattccgagaagctggacttgaagagcaaccctgctgggatcgtccccattgagatcgaaggttgcgagaacatcggtaccgacaagctgtgccagctggatacttttcagaagcgtgttgcccaggtcattgagcccgcgtgccaaatctaatagcacgaacctctctgaaggaggttctgagacacgcgcgattcttctgtatatagttttatttttcactctggagtgcttcgctccaccagtacataaacctttttttcacgtaacaaaatggcttcttttcagaccatgtgaaccatcttgatgccttgacctcttcagttctcactttaacgtagttcgcgtttgtctgtatgtcccagatcatttaaactcgacgtaataatgggcgctttctactctggacttgcctctttgtgctttacattgggggtcgagcctttccaatcagaagatgcacttaccaaagtagcgctaggctacacagcgctttacccccgaatcaggcgtggggtcactaccccgacgcccttttttttttttttttttgtattcacagagcggtttgcacaatctagatttgttcctcccgcctagtcggtcgccttgtacttggcccggtactcctccgtcctcctgatgtcctcctcggatgcgagccccttgataccctcgatgatgtcgtccaaggtgagaatcgcaaagatgggcagaccatactccttccggagctcaccgatcgcgctcggcatcggcttgctatcgtcgccatccggcgaaggcagcttctccatgcggtccagcgcgaccacgatgccggctacgataccgccctccttctcaatcttggcgatcgcctcacgcttcgccgtgccagccgtgatcacatcatccacgataagcaccctcttgcccttgagcggcgcccccacaatgttgccgccctcaccgtggtcctttgcctccttgcggtcgaacgagtagcacgtggtatggccgtacgtggccggatccagctggcccagccgaatggtggccgccgtcgccaacgggatccccttgtaggccgggccgaagacgatgtcgaactggatcgcgccgctccggtgcgcctcgatgatggcgtgcgcgtacgccgtcgcgagcgcctcgagcagatcggcccggtagaagtctcccgcgttgaagaagtagggcgagatgcgcttcgacttgagctcgaagctgccaaacttgaggatgttgccggcgatggccgcccggaggaagtcggctttgtaagaagcgagtggggccatgtctgtcggttcgtacgtgtcttacgggttcttgggctggaagtttatttgttaccggagggaactcctggtgaagattcgaaaaaaagaaatgagggagttttgtcaaacgtccccctatcgcagagctgaagagagcccctccatcgcgaatttcttggtggggttggattcatggtggggcccaagggtcgttaaacttttttgtatgtctacggagttacacagtactccgtacgtcgtaccccacttgcaatgcttctcgagctgggcttgggatattttgataacgcacaccgcctgccttatcttatcattttgtgctctccgcaaaaaattatttcgggcgatagccgaggctctctgaaggaggttctgagacacgcgcgattcttctgtatatagttttatttttcactctggagtgcttcgctccaccagtacataaacctttttttcacgtaacaaaatggcttcttttcagaccatgtgaaccatcttgatgccttgacctcttcagttctcactttaacgtagttcgcgtttgtctgtatgtcccagttgcatgtagttgagataaatacccctggaagtgggtctgggcctttgtgggacggagccctctttctgtggtctggagagcccgctctctaccgcctaccttcttaccacagtacactactcacacattgctgaactgacccatcataccgtactttatcctgttaattcgtggtgctgtcgactattctatttgctcaaatggagagcacattcatcggcgcagggatacacggtttatggaccccaagagtgtaaggactattattagtaatattatatgcctctaggcgccttaacttcaacaggcgagcactactaatcaacttttggtagacccaattacaaacgaccatacgtgccggaaattttgggattccgtccgctctccccaaccaagctagaagaggcaacgaacagccaatcccggtgctaattaaattatatggttcatttttttaaaaaaattttttcttcccattttcctctcgcttttctttttcgcatcgtagttgatcaaagtccaagtcaagcgagctatttgtgctatagctcggtggctataatcagtacagcttagagaggctgtaaaggtatgataccacagcagtattcgcgctataagcggcactcctagactaattgttacggtctacagaagtaggtaataaaagcgttaattgttctaaatactagaggcacttagagaagctatctaaatatatattgaccctagcttattatccctattagtaagttagttagctctaacctatagatagccaaatgctataataggtaccagggttcaaaaggaaaaatgacgacccgttataacttagtaaatatagtcttttatctattatgaccgtccttacaatcgttcctctatcctacggtcgtatctatccgcttatccgtcagtccttcttactactagccaggcctaggttgtctaggaatacggatagtgctgacgggcgctcctactgtaggtgtacgagagtcctagctctggcctaggtgtccctggctagggtacggttactatagagcgcgctagtagccctcttacgtgaggggctctcctatgctagcgtttctagccgtagtcgtttctagctagtagtcgtaggcggtaggtatacgactaggtaggcgattaagctaggaggggcgctagcccttatagtagctagtggtagctaagctataggtagttacgttctaggagccctttatagtatagtggctaatatagtatgcttactaagttaggtaattatacctttaagctattatagcttcttatttacgcccttcttctagttaagggtagctatatagctattatttatagctattaaagtctttcatagctgcttaagctcttccttaatctagttaagggccttagtaatgcgatcatttaaatacgtagc

SEQ ID NO: 3 - DNA, 7599 bp, synthetic construct
  source 1..7599: mol_type = other DNA; plasmid = Plasmid MT940
ctagtcatatgacgcgtggtaccgggcccgacgtcaggcctctcgagatttaaatcgagcggccgcggccggcctttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgggccggccggcgcgccgctagcgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccgctagcggatcatttaaatcccgggtctagaggatcccaattgttaattaacgcagaagagcatcgatgaggtatccgatttggggaacgtcgatgaaagtattgcaaaagtgacgagagttgcgcaactaactcgctgccgaagaagctgcggaagaaagagaacaccgaaagtggaataacgttacggatgtcctgacctcaaagttgaaaccagcccttcctgctctatttgggaaagcggcttgcccttgaatgcgctgcactgtggcacgactaccagtgatcgggaggagcaaactaccctggtccgttccttggtggggcggcactaggcccaacttagggtgatcggaggtcgatgccgcggtcctcgttggtctgggctcttctcatttcccggtttgcaccccccgttgcacctgctgatcgcccgccaacgccgatgaggttgcgcccagaccgacaatcaccgcggctgcattcccaagtatattgaagatggcaccaggtacccggttttgcgtcccagtcgtttggtgccaaatttgggagtttttgagcctcaagatctggggaaatcgacctcaacttccatacaagttaaagtcgcacacacggcgagttccacgaagagacacatttttttctgaaggcctctctccccgcacatcagaaaccaccaaataccaagactgcagaagccggggtaagtgggccaccgggactacactaaaatgcggggagaagcgagatccgttgcgaagggaagggatggggtgtgctgcggctttctccgctctcgtgcgccttttgcttgaatctagtgtacaccagggtaggctccgaaggagtatctacggcagcgctgttcgtgctgcgttgagagtcagggcggagacgagcaggcgacaggagcctcgcaccggcacttcggatcgcatttgcgcggagcgtcaaatacgctcttctgcggtcatcagagagcatcgtgaaccaaggttcttccgcagggcggcctgggcttcgcagagtcgcactcggcggacgccttccgtgtcacccctgataacctggctgccgcgcccagactcctccaatgaggtgtgtggttgccctcgccgacccttcagcaaccttaatcgcttccatcgcacggctccacgtcctcgaacgatgccctcagtccgtgcccggccgtggcaaccataacgtgacatcgccgcccagcctactagccgctatcgaccggttaggcttgtcaccgcagcgcccattctccatcgggcctctactctgatccacctcacccaccgcaagcactagcgagcctcaccagagtgcaagcgacacgacccgcttggcccttcgtccttgactatctcccagacctcttgccatcttgccgacgccgcccccttttttttctcctccccctgccggcaggtcggtggccccagtcccgagatggcattgctccgttgtccatgacgacccatcattcgatggctgactggcacactcgtcttgtacgtagatcagagacccaataggccgaaatcggcaaaatcccttataaaatagggcgaacaaaagaatagaccggataaaacgaaaggcccagtctttcgactgagcctttcgttttattggtagcagacaagcccgtcagggcgcgtcagcgggtgttggcgggtgtcggggctggctttactatgcggcatcagagcagattgtactgagagtgcaccagatgcggtgtgaaataccgcacagatgcgtaaggagaaaataccgcatcagacgccattcgccattcaggctgcgcaactgttgggaagggcgatctgtgcgggcctcttcgctattacgccaactggcgaaagggggatgtgctgcaaggcgattaagttgggtaacgccagggttttcccagtcacgacgttgtaaaacgacggccagtgagcgcgcgtaatacgactcactggaactccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaaggtctcatcgagcggccgctacgtataacctagccttcaaacttacgaaaggatgatgtcgataaactcctgtgccctgtacgaaaatgatcagcttagttcaggaaaggaacagcatatatacgaattgaacccttacgtaggcacaggcgcgatatccttccaggccatggcaggcaacgcgcagggtgctgtccgaatttggcgatgggggtgtttgggagttgtcagatagggagcacctggcggggcggtgtcgatttgccgtccccaagtcgcccgaaataattttttgcggagagcacaaaatgataagataaggcaggcggtgtgcgttatcaaaatatcccaagcccagctcgagaagcattgcaagtggggtacgacgtacggagtactgtgtaactccgtagacatacaaaaaagtttaacgacccttgggccccaccatgaatccaaccccaccaagaaattcgcgatggaggggctctcttcagctctgcgatagggggacgtttgacaaaactccctcatttctttttttcgaatcttcaccaggagttccctccggtaacaaataaacttccagcccaagaacccgtaagacacgtacgaaccgacagacatggccccactcgcttcttacaaagccgacttcctccgggcggccatcgccggcaacatcctcaagtttggcagcttcgagctcaagtcgaagcgcatctcgccctacttcttcaacgcgggagacttctaccgggccgatctgctcgaggcgctcgcgacggcgtacgcgcacgccatcatcgaggcgcaccggagcggcgcgatccagttcgacatcgtcttcggcccggcctacaaggggatcccgttggcgacggcggccaccattcggctgggccagctggatccggccacgtacggccataccacgtgctactcgttcgaccgcaaggaggcaaaggaccacggtgagggcggcaacattgtgggggcgccgctcaagggcaagagggtgcttatcgtggatgatgtgatcacggctggcacggcgaagcgtgaggcgatcgccaagattgagaaggagggcggtatcgtagccggcatcgtggtcgcgctggaccgcatggagaagctgccttcgccggatggcgacgatagcaagccgatgccgagcgcgatcggtgagctccggaaggagtatggtctgcccatctttgcgattctcaccttggacgacatcatcgagggtatcaaggggctcgcatccgaggaggacatcaggaggacggaggagtaccgggccaagtacaaggcgaccgactaggcgggaggaacaaatctagattgtgcaaaccgctctgtgaatacaaaaaaaaaaaaaaaaaagggcgtcggggtagtgaccccacgcctgattcgggggtaaagcgctgtgtagcctagcgctactttggtaagtgcatcttctgattggaaaggctcgacccccaatgtaaagcacaaagaggcaagtccagagtagaaagcgcccattattacgacgaacctctctgaaggaggttctgagacacgcgcgattcttctgtatatagttttatttttcactctggagtgcttcgctccaccagtacataaaccttttttttcacgtaacaaaatggcttcttttcagaccatgtgaaccatcttgatgccttgacctcttcagttctcactttaacgtagttcgcgtttgtctgtatgtcccagttgcatgtagttgagataaatacccctggaagtgggtctgggcctttgtgggacggagccctctttctgtggtctggagagcccgctctctaccgcctaccttcttaccacagtacactactcacacattgctgaactgacccatcataccgtactttatcctgttaattcgtggtgctgtcgactattctatttgctcaaatggagagcacattcatcggcgcagggatacacggtttatggaccccaagagtgtaaggactattattagtaatattatatgcctctaggcgccttaacttcaacaggcgagcactactaatcaacttttggtagacccaattacaaacgaccatacgtgccggaaattttgggattccgtccgctctccccaaccaagctagaagaggcaacgaacagccaatcccggtgctaattaaattatatggttcattttttttaaaaaaatttttttcttcccattttcctctcgcttttctttttcgcatcgtagttgatcaaagtccaagtcaagcgagctatttgtgctatagctcggtggctataatcagtacagcttagagaggctgtaaaggtatgataccacagcagtattcgcgctataagcggcactcctagactaattgttacggtctacagaagtaggtaataaaagcgttaattgttctaaatactagaggcacttagagaagctatctaaatatatattgaccctagcttattatccctattagtaagttagttagctctaacctatagatagccaaatgctataataggtaccagggttcaaaaggaaaaatgacgacccgttataacttagtaaatatagtcttttatctattatgaccgtccttacaatcgttcctctatcctacggtcgtatctatccgcttatccgtcagtccttcttactactagccaggcctaggttgtctaggaatacggatagtgctgacgggcgctcctactgtaggtgtacgagagtcctagctctggcctaggtgtccctggctagggtacggttactatagagcgcgctagtagccctcttacgtgaggggctctcctatgctagcgtttctagccgtagtcgtttctagctagtagtcgtaggcggtaggtatacgactaggtaggcgattaagctaggaggggcgctagcccttatagtagctagtggtagctaagctataggtagttacgttctaggagccctttatagtatagtggctaatatagtatgcttactaagttaggtaattatacctttaagctattatagcttcttatttacgcccttcttca

SEQ ID NO: 4 - protein, 322 aa, Glycine max
  source 1..322: note = Accession Version: XP_003552015.1
MKRLSSSDSSSALMTICPSTEEHSPRNSQHMYGREFQSMLDGLDEEGCVEEPGYQSEKKRRLSVDQVKALEKNFEVENKLEPERKVKLAQELGLQPRQVAVWFQNRRARWKTKQLERDYGVLKANYDALKLNFDTLDQDNEALRKQVKELKSRLLQEENTGGSGVSVKEEIITRPADSEDKTMEQSKSDPSSETSNINPSSESEEDHLNYECFNNNDDCVGGTAASLLQVDFKDGSSDSDGSSAILNEDNMYSPLKFNNCSISTSPSSSSMMNCFQFQKPYHHHAQYVKMEEHNFLSADEACNFFSDEQAPTLQWYCPEQWS

SEQ ID NO: 5 - DNA, 2595 bp, synthetic construct
  source 1..2595: mol_type = other DNA; note = Cloning vector MT944
ggccgcatcgaattcctgcagcccgggggatccgcccgggctaggcgccgagcggccgccaccgcggtgagctctcgatcggtcgtgctatgctagttattgctcagaatcagctgcattaatgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtatgcatctagtgcttggattctcaccaataaaaaacgcccggcggcaaccgagcgttctgaacaaatccagatggagttctgagttaattaatggatctatcaacaggagtccaagcaagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccgcccacagtcgatgaatccagaaaagcggccattttccaccatgatattcggcaagcaggcatccccatgtgtcacgacgagatcctcgccgtcgggcatacgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatctgatcttgatcccctgcgccatcagattcttggcggcaagaaagccatccagtttactttgcagggcttcccaaccttaccagagggcgccccagctggcaattccgacgtttagctgcggccgctacgtagtttaaactcgatgagacctttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagttccagtgagtcgtattacgcgcgctcactggccgtcgttttacaacgtcgtgactgggaaaaccctggcgttacccaacttaatcgccttgcagcacatccccctttcgccagttggcgtaatagcgaagaggcccgcacagatcgcccttcccaacagttgcgcagcctgaatggcgaatggcgtctgatgcggtattttctccttacgcatctgtgcggtatttcacaccgcatctggtgcactctcagtacaatctgctctgatgccgcatagtaaagccagccccgacacccgccaacacccgctgacgcgccctgacgggcttgtctgctaccaataaaacgaaaggctcagtcgaaagactgggcctttcgttttatccggtctattcttttgttcgccctattttataagggattttgccgatttcggcctattgggtctctgatcatttaaatacgtagc

SEQ ID NO: 6 - DNA, 4723 bp, synthetic construct
  source 1..4723: mol_type = other DNA; plasmid = Deletion plasmid MT2013
gctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgctcgatttaaatctttcaacaccacaacactgaccgggttcatttggtggttccgcatccagaatgcggggtgtactagtatcctgttgagacaagggactggctgcatcgggaaatcgatgcttggcttgctgcggccagcccggtcgccactgatgagtggctgtcgacttgccaagggggaaacgtagcagagagagcaacaccaatcgcgcaaagatctcgggggcttacggagtacaccaaagagagagattccgtcgcatcctgatatccatactgggtgtatgccacagcctccggcaatccgcacgttgaagcagagcaccacaggaaagccacccatagcgaactgaaaagtgccgacaggaacccgtggcggccgagcggacgcagacatacgtcctgtacggcttacatgtgcatactctctagatatactctgatcgactgcagagtgtaacggcgaacagagggtgctcagaaggcggcccaccagacaaagaccgacctggcaaggaataaagacaagccgagcagacgaacaggactgtttcagttgcgggtggttgaacgcgtgggtaaggacggtcggcagactggccaccagggtgctgaccttggggtatcgaatcgatactcacctactgtcggtacgctactgtggtacgctgttgcgcgaggtttctgcttgcccgcgggatgcaagtcgaggcccggccgctcttcaagcatacatcaagctttggagttctctcactcgcttacccctgtccactagccacaccaaccacgggcgcggaccgatggtgggtggtacaaggacgtgagtgaactggcgtgcgggcgcaggacttctctctcttttttttttcgcagagggcgccaatatacacgtaccaatgtacctacgtgccttgggtcacatcgaatcggaaacccctgtcgacctacacgacgacacccgaagcccaaacgcagccccatgccacccaagcatccacggtatccctgtaccgcccccctgccccctgacaaccctgacgtccgaagtagtgtacgaagtactccgtaccgcccatgcaatgtgcggactgagcctcgccctactgggaaagcccaagaaacgatccgccctccaccagggcccggccgacgaagatgggcgccggggggtatatagaaaacctcaccccaaggctggcccgtccgtctcccactcgttttgcattaccgtcaccaacaacaaccaagagagcttttttttcttcacaaacccctcgcgccccatacagtcgcactcggggctcgcaacagctctcgagtttgaccgtctcctctctgggaatcccaaggctttttttcccccttcagagcacagcctgcaagagcgattaagaatctgggtatgcgggtgtaaaggaaggtcagttgctggcgatttaggtgggctgcaagtagagggtgactgacgctcttgctgtcccccagtcttcgaggttgcgcgcggctttaccaacatcccttaaagagaaggccagcgaggcctctcctctcctctcacccaccctagggataacagggtaattgaattcgaagttcctatactttctagagaataggaacttccgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatggccaccctcgatgacacggcttaccgctaccgtaccagcgtccccggcgacgccgaagccatcgaggccctggatggctctttcaccacggataccgtctttcgcgttaccgctaccggtgacggcttcacgctccgtgaggtgcccgtcgaccctcccctgaccaaggttttccctgatgacgaatctgacgatgaatccgacgatggcgaggacggcgatcccgactctcgcacgttcgtcgcttacggcgatgacggtgacctggccggctttgtcgttgtgtcctattccggttggaaccgtcgcctgaccgtcgaagacatcgaggttgcccccgagcatcgcggccacggtgtcggccgcgctctcatgggtctcgccacggagttcgcccgtgagcgcggcgccggtcacctctggctggaggttaccaatgtgcggccgctacgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctct

SEQ ID NO: 7 - DNA, 4626 bp, synthetic construct
  source 1..4626: mol_type = other DNA; plasmid = Deletion plasmid MT2014
ggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgggccggccggcgcgccgctagcgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccgctagcgatttaaatcccgtagcggccgcttagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacctaagcattcatggccaccctcgatgacacggcttaccgctaccgtaccagcgtccccggcgacgccgaagccatcgaggccctggatggctctttcaccacggataccgtctttcgcgttaccgctaccggtgacggcttcacgctccgtgaggtgcccgtcgaccctcccctgaccaaggttttccctgatgacgaatctgacgatgaatccgacgatggcgaggacggcgatcccgactctcgcacgttcgtcgcttacggcgatgacggtgacctggccggctttgtcgttgtgtcctattccggttggaaccgtcgcctgaccgtcgaagacatcgaggttgcccccgagcatcgcggccacggtgtcggccgcgctctcatgggtctcgccacggagttcgcccgtgagcgcggcgccggtcacctctggctggaggttaccaatgtcaacgctcccgccattcacgcctaccgtcgcatgggctttaccctctgcggcctggataccgctctctacgatggcaccgcctccgatggcgagcaggccctctatatgagcatgccttgcccctgagaagttcctatactttctagagaataggaacttcgggcccacccgggtctagagagggatctctcatctgctgccgggtgcgttcgatggcatgagtgcgcacctaacccaccctcccgaacgaacctcctcatcctcctttttgataccccgatgaccgaacacttccctatactattcatgcatactcgtgacataaaatcacgcttgtataccattaatgtgtttttttcttcttcacttcttcctgctctctctctcgcgcgcgcttccggcgaaccacatcggacacgggctggattcgcatcgaaatgaaagtcgagatacacaaaccgggacgaagactctccaaggtctagttttcttttcttttcttttctcagtggcggcgacgtggtgtgtgtgtgaggtggattgggactttgggttcattatatgtttgtggcgcgtgtggatctttcattggacttttaatctttctcacttccttccatcttcttgcttccaactctcagacctcggaatactcactggggcggaacggacggacggacggacggatctaataatgattgcatgaccatgggatcgacattggcttggatgaaataggaccagggaaatgggagaagaatatacaaggcccggacatacttttttgcggtttacttctttagaatattctttttgttgtcctccgttcactctcgaggtttctttcctcttccgccggtctgcttgcttgatacattgcgcggtggatagataatcgataccctgttctcatcaaaccatcttctacgcgttcccgcctgtgttgtgatgatttttgtggggccaatgacggtgaaacggtaggttatatacaaggcattcggaaggagtctggaagcgagtacatgcttatctagctttgtgtggccctcactgtcggcctcttgatgagtgtttatttggtgtttggcggctgccgattttgcagtcgatacagaaaaatcagatgcagtgctgttagtaagcgagtatgagtatgtatgtactgtacgttcatctctgatcttccattcccgacttgacttccatgcgtaaggctgctgcacccacgattttcttttcttttcttttctttctttctttctttctttcttttttttttttttctttttttttgttttttgcgccagttgatcggggccctcagaaacgcccactgccatgaagtgttctcggagcacttccgagcaacttgcctcattgctccacaagatctccgatgacttctacaaggttcggtggggaccagcccgtcaccgggatataggttggagcgatgcacagggcctttctgagtgttgcccgcgggacaaagtctattctcgtcttgagtggcgacgcgaccagtgtgggttcggcatttgccagtttggagggaaaggattcaacagcttatccaaacacgcacggcacgcacttggaccggctcgatattcctttcccactttgctcccaaacgttgggctcccagggtctgacctgccctcacaacccacccactcctaaggcctcttttgcccacctttacttgttagatttaaatcgagcggccgcggccggcctttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccaggg

(19) United States
(12) Patent Application Publication
(10) Pub. No.: US 2026/0193625 A1
(43) Pub. Date: Jul. 9, 2026

(54) METHODS OF REDUCING CONIDIATION IN CELL CULTURE

(71) Applicants: BASF CORPORATION, Florham Park, NJ (US); THE REGENTS OF THE UNIVERSITY OF CALIFORNIA, Oakland, CA (US)

(72) Inventors: Stefan Haefner, Ludwigshafen (DE); Katja Gemperlein, Florham Park, NJ (US); Louise N. Glass, Berkeley, CA (US); Yang Li, Oakland, CA (US); Lori Huberman, Oakland, CA (US); Florian Drescher, Oakland, CA (US)

(21) Appl. No.: 19/128,252

(22) PCT Filed: Nov. 15, 2023
(86) PCT No.: PCT/US2023/079777

§ 371 Date: May 8, 2025

Related U.S. Application Data
(60) Provisional application No. 63/383,798, filed on Nov. 15, 2022.

(51) Int. Cl. C12N 9/16; C12N 1/14; C12P 21/02; C12R 1/645

(52) U.S. Cl. CPC C12N 9/16; C12N 1/14; C12P 21/02; C12R 2001/645; C12Y 301/03008

(57) ABSTRACT

Provided herein is a method of reducing conidiation during cell culture of a T. thermophilus strain, the method comprising growing, in a submerged cell culture, a variant T. thermophilus strain that has reduced res-1 activity as compared to a wild-type T. thermophilus strain.

### BACKGROUND

T. thermophilus is used in biotechnological enzyme production and has been proposed for a variety of biotechnological applications including biofuel production (Grieco et al., Frontiers in Bioeng. Biotechnol. 8:1-11, 2020; Singh, B., Critic. Rev. Biotechnol., 36:59-69, 2016). However, the ability of T. thermophilus to form conidia under fermentation conditions poses an environmental contamination risk and possible work safety risks when handling suspensions during and after harvesting. This is both true for state-of-the-art bioreactors in enclosed facilities with reduced contact to the outside environment and for bioreactors not enclosed in facilities as used for example in bioethanol production. Thus, there remains a need in the art for a method of culturing T. thermophilus that greatly reduces contamination risks by preventing conidiation of T. thermophilus.

### SUMMARY

In one aspect, provided herein is a method of reducing conidiation during cell culture of a T. thermophilus strain, the method comprising growing, in a submerged cell culture, a T. thermophilus strain that has reduced res-1 activity as compared to a wild-type T. thermophilus strain.

In another aspect, provided herein is a method of increasing production of a heterologous protein in a T. thermophilus strain, the method comprising growing a T. thermophilus strain comprising a nucleic acid encoding a heterologous protein in a submerged culture, wherein the T. thermophilus strain has reduced res-1 activity, wherein the expression level of the heterologous protein produced by the T. thermophilus strain that lacks res-1 activity is higher than an expression level of the heterologous protein when produced by a T. thermophilus strain having wild-type res-1 activity.

### BRIEF DESCRIPTION OF THE FIGURES

FIGS. 1A and 1B. Evaluation of the phenotype of T. thermophilus Δres-1 variant. FIG. 1A is a graph showing the number of conidia produced in submerged cultures of wild-type T. thermophilus, T. thermophilus Δres-1 variant, and the T. thermophilus Δres-1 complemented strain (Δres-1 C). FIG. 1B is a graph showing the amount of dried fungal biomass obtained from submerged cultures inoculated with 106 conidia from WT, the Δres-1 strain and the Δres-1 C strain grown for 72 hours.

FIGS. 2A and 2B are microscopic pictures from end of cultivation samples. FIG. 2A is a picture of the parental strain SB1221. FIG. 2B is a picture of the Res-1 deletion strain T401

FIGS. 3A and 3B are microscopic pictures of fermentation broth at the end of a glucose limited fed batch fermentation. FIG. 3A is a picture of parental strain SB1221.

FIG. 3B is a picture of the Res-1 deletion strain T401.

### DETAILED DESCRIPTION

The present disclosure is based, in part, on the discovery that conidiation during cell culture of a T. thermophilus strain can be reduced by growing, in a submerged cell culture, a variant T. thermophilus strain that has reduced res-1 activity as compared to a wild-type T. thermophilus strain.

In one aspect, described herein is a method of reducing conidiation during cell culture of a T. thermophilus strain, the method comprising growing, in a submerged culture, a variant T. thermophilus strain that has reduced res-1 activity as compared to a wild-type T. thermophilus strain. In some embodiments, decreased activity (or expression) of a res-1 gene is achieved by a deactivation, mutation or knock-out of the res-1 gene. This could be done by deletion of part or total of the coding region and/or the promoter of the gene, by mutation of the gene such as insertion or deletion of a number of nucleotides for example one or two nucleotides leading to a frameshift in the coding region of the gene, introduction of stop codons in the coding region, inactivation of the promoter of the gene by for example deleting or mutating promoter boxes such as ribosomal entry sides, the TATA box and the like. The decrease may also be achieved by degrading the transcript of the gene for example by means of introduction of ribozymes, dsRNA, antisense RNA or antisense oligonucleotides. The decrease of the activity of a gene may be achieved by expressing antibodies or aptamers in the cell specifically binding the target enzyme. Other methods for the decrease of the expression and/or activity of a gene are known to a skilled person.

In some embodiments, the reduced activity (or expression) of the res-1 gene is achieved by introduction of a mutation into the gene, preferably a deletion. Any number of nucleotides can be deleted. In some embodiments, a deletion involves the removal of at least one, at least two, at least three, at least four, at least five, at least ten, at least fifteen, at least twenty, or at least 25 nucleotides. In some embodiments, a deletion involves the removal of 10-50, 25-75, 50-100, 50-200, or more than 100 nucleotides. In some embodiments, a deletion involves the removal of an entire target gene, e.g., a res-1 gene.

### Heterologous Proteins

In some embodiments, the T. thermophilus variant described herein further comprises a nucleic acid encoding a heterologous protein. Exemplary heterologous proteins include, but are not limited to, an enzyme, an enzyme, a structural protein or a storage protein. In some embodiments, the heterologous protein is an enzyme. Exemplary enzymes include, but are not limited to, phytase, hydrolase, isomerase, ligase, lyase, oxidoreductase, transferase, aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, nuclease, oxidase, pectinolytic enzyme, peroxidase, phosphodiesterase, polyphenoloxidase, proteolytic anzyme, ribonuclease, transglutaminase, xylanase, and beta-xylosidase. In some embodiments, the heterologous protein is phytase.

In another aspect, provided herein is a method of increasing production of a heterologous protein in a T. thermophilus strain, the method comprising growing a T. thermophilus strain comprising a nucleic acid encoding a heterologous protein in a submerged culture, wherein the T. thermophilus strain has reduced res-1 activity, and wherein the expression level of the heterologous protein produced by the T. thermophilus strain that lacks res-1 activity is higher than an expression level of the heterologous protein when produced by a T. thermophilus strain having wild-type res-1 activity. In some embodiments, the expression level of the heterologous protein produced by the T. thermophilus strain having reduced res-1 activity is at least 5% higher than the heterologous protein when expressed by a T. thermophilus strain having wild-type res-1 activity. In some embodiments, the expression level of the heterologous protein produced by the T. thermophilus strain having reduced res-1 activity is at least 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, or 70% higher than the heterologous protein when produced by a T. thermophilus strain having wild-type res-1 activity.

In some embodiments, the T. thermophilus variant described herein is transformed or transfected with a vector comprising the nucleic acid encoding the heterologous protein. The term “vector”, preferably, encompasses phage, plasmid, viral or retroviral vectors as well as artificial chromosomes, such as bacterial or yeast artificial chromosomes. The vector may comprise selectable markers for propagation and/or selection in a host. The vector may be incorporated into a host cell by various techniques well known in the art. If introduced into a host cell, the vector may reside in the cytoplasm or may be incorporated into the genome. In the latter case, it is to be understood that the vector may further comprise nucleic acid sequences which allow for homologous recombination or heterologous insertion. Vectors can be introduced into prokaryotic or eukaryotic cells via conventional transformation or transfection techniques. The terms “transformation” and “transfection”, conjugation and transduction, as used in the present context, are intended to comprise a multiplicity of prior-art processes for introducing foreign nucleic acid (for example DNA) into a host cell, including calcium phosphate, rubidium chloride or calcium chloride co-precipitation, DEAE-dextran-mediated transfection, lipofection, natural competence, carbon-based clusters, chemically mediated transfer, protoplast transformation, electroporation or particle bombardment (e.g., “gene-gun”). Suitable methods for the transformation or transfection of host cells, including plant cells, can be found in Sambrook et al. (Molecular Cloning: A Laboratory Manual, 2nd ed., Cold Spring Harbor Laboratory, Cold Spring Harbor Laboratory Press, Cold Spring Harbor, NY, 1989) and other laboratory manuals, such as Methods in Molecular Biology, 1995, Vol. 44, Agrobacterium protocols, Ed.: Gartland and Davey, Humana Press, Totowa, New Jersey. Alternatively, a plasmid vector may be introduced by heat shock or electroporation techniques.

Preferably, the vector referred to herein is suitable as a cloning vector, i.e. replicable in microbial systems. Such vectors ensure efficient cloning in bacteria and, preferably, yeasts or fungi. These vector systems, preferably, also comprise further cis-regulatory regions such as promoters and terminators and/or selection markers with which suitable transformed host cells or organisms can be identified.

Vectors and processes for the construction of vectors which are suitable for use in fungi, such as the filamentous fungi, comprise those which are described in detail in: van den Hondel, C. A. M. J. J., & Punt, P. J. (1991) “Gene transfer systems and vector development for filamentous fungi, in: Applied Molecular Genetics of fungi, J. F. Peberdy et al., Ed., pp. 1-28, Cambridge University Press: Cambridge, or in: More Gene Manipulations in Fungi (J. W. Bennett & L. L. Lasure, Ed., pp. 396-428: Academic Press: San Diego).

In some embodiments, the vector (or vectors) described herein comprising the expression cassette are propagated and amplified in T. thermophilus. In some embodiments, one copy of the vector is propagated and amplified in T. thermophilus. In some embodiments, two or more (e.g., 3, 4, 5, 6 7, 8 or more) copies of the vector are propagated and amplified in T. thermophilus.

In some embodiments, the vector comprises a nucleic acid encoding the heterologous protein is operably linked to a promoter. The terms “operably-linked” or “functionally linked” refer to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

The term “promoter” as used herein refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised, in some cases, of a TATA box and other sequences that serve to specify the site of transcription initiation, to which regulatory elements are added for enhancement of expression. “Promoter” also refers to a nucleotide sequence that includes a minimal promoter plus regulatory elements and that is capable of controlling the expression of a coding sequence or functional RNA. This type of promoter sequence consists of proximal and more distal upstream elements, the latter elements often referred to as enhancers. Accordingly, an “enhancer” is a DNA sequence, which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements, derived from different promoters found in nature, or even be comprised of synthetic DNA segments.

A promoter may also contain DNA sequences that are involved in the binding of protein factors, which control the effectiveness of transcription initiation in response to physiological or developmental conditions. The “initiation site” is the position surrounding the first nucleotide that is part of the transcribed sequence, which is also defined as position +1. With respect to this site all other sequences of the gene and its controlling regions are numbered. Downstream sequences (i.e., further protein encoding sequences in the 3′ direction) are denominated positive, while upstream sequences (mostly of the controlling regions in the 5′ direction) are denominated negative. Promoter elements, such as a TATA element, that are inactive or have greatly reduced promoter activity in the absence of upstream activation are referred as “minimal” or “core” promoters. In the presence of a suitable transcription factor, the minimal promoter functions to permit transcription. A “minimal” or “core” promoter thus consists only of all basal elements needed for transcription initiation, e.g., a TATA box and/or an initiator.

The expression of the polynucleotide encoding the heterologous protein of interest can be determined by various well known techniques, e.g., by Northern Blot or in situ hybridization techniques as described in WO 02/102970, the disclosure of which is incorporated herein by reference in its entirety.

### Cell Culture

In some embodiments, the variant T. thermophilus having reduced res-1 activity described herein are grown in a submerged cell culture under batch or continuous fermentation conditions. Classical batch fermentation is a closed system, wherein the compositions of the medium is set at the beginning of the fermentation and is not subject to artificial alterations during the fermentation. A variation of the batch system is a fed-batch fermentation. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. Continuous fermentation is a system where a defined fermentation medium is added continuously to a bio-reactor and an equal amount of conditioned medium (e.g., containing the desired end-products) is removed simultaneously for processing. Continuous fermentation generally maintains the cultures at a constant high density where cells are primarily in the growth phase where production of end products is enhanced. Continuous fermentation systems strive to maintain steady state growth conditions. Methods for modulating nutrients and growth factors for continuous fermentation processes as well as techniques for maximizing the rate of product formation are well known in the art of industrial microbiology.

In some embodiments, fermentations are carried out in a temperature within the range of from about 10° C. to about 60° C., from about 15° C. to about 50° C., from about 20° C. to about 45° C., from about 25° C. to about 45° C., from about 30° C. to about 45° C. and from about 35° C. to about 42° C. In some embodiments, the temperature is about 36° C., 37° C. or 38° C. In some embodiments, the temperature is about 39° C. or 40° C.

In some other embodiments, the fermentation is carried out for a period of time within the range of from about 8 hours to 240 hours, from about 16 hours to about 216 hours, from about 24 hours to about 196 hours, from about 36 hours to about 172 hours, or Preferably the fermentation is carried out from about 48 hours to about 168 hours.

In some other embodiments, the fermentation is carried out at a pH in the range of about 4 to about 9, in the range of about 4.5 to about 7.5, in the range of about 5 to about 7. In some embodiments, the fermentation will be carried out in the range of about 5.5 to about 6.5.

In some embodiments, the variant T. thermophilus strain described herein is grown under carbon-limited conditions. In some embodiments, the variant T. thermophilus strain described herein is grown under glucose-limited conditions. By “limited glucose conditions” is meant that the amount of glucose that is added is less than or about 105% (such as about 100%) of the amount of glucose that is consumed by the cells. In particular embodiments, the amount of glucose that is added to the culture medium is approximately the same as the amount of glucose that is consumed by the cells during a specific period of time. In some embodiments, the rate of cell growth is controlled by limiting the amount of added glucose such that the cells grow at the rate that can be supported by the amount of glucose in the cell medium. In some embodiments, glucose does not accumulate during the time the cells are cultured. In various embodiments, the cells are cultured under limited glucose conditions for greater than or about 1, 2, 3, 5, 10, 15, 20, 25, 30, 35, 40, 50, 60, or 70 hours. In various embodiments, the cells are cultured under limited glucose conditions for greater than or about 5, 10, 15, 20, 25, 30, 35, 40, 50, 60, 70, 80, 90, 95, or 100% of the total length of time the cells are cultured.

In some embodiments, the methods described herein further comprise separating the variant T. thermophilus from the fermentation broth (the so called “biomass”). Processes for removing the biomass are known to those skilled in the art, and comprise filtration, sedimentation, flotation or combinations thereof. Consequently, the biomass can be removed, for example, with centrifuges, separators, decanters, filters or in a flotation apparatus. The selection of the method is dependent upon the biomass content in the fermentation broth and the properties of the biomass, and also the interaction of the biomass with the protein of interest (i.e., the product of value). In one embodiment, the fermentation broth can be sterilized or pasteurized. In a further embodiment, the fermentation broth is concentrated. Depending on the requirement, this concentration can be done batch wise or continuously. The pressure and temperature range should be selected such that firstly no product damage occurs, and secondly minimal use of apparatus and energy is necessary. The skillful selection of pressure and temperature levels for a multistage evaporation in particular enables saving of energy.

The recovery process may further comprise additional purification steps in which the fermentation product is further purified. If, however, the fermentation product is converted into a secondary organic product by chemical reactions, a further purification of the fermentation product might, depending on the kind of reaction and the reaction conditions, not necessarily be required. For the purification of the fermentation product obtained in process step II) methods known to the person skilled in the art can be used, including, but not limited to chromatography, precipitation, crystallization, filtration, electrodialysis. The resulting solution may be further purified by means of ion exchange chromatography in order to remove undesired residual ions.

All patent documents and non-patent literature referenced herein is incorporated herein by reference.

### EXAMPLES

### Example 1—Conidiation in Submerged and Liquid Culture of T. thermophilus res-1 Deletion Variant

T. thermophilus variants comprising a res-1 deletion were generated by double-joint PCR deletion (Yu et al., Fungal Genet. Biol., 41:973-981, 2004) using the resistance gene hygromycin phosphotransferase as a selectable marker.

Deletion mutants T. thermophilus res-1 were confirmed by PCR using primers specific for the hygromycin knockout cassette and sequences flanking the T. thermophilus target genes. Results showed that the T. thermophilus Δres-1 variant failed to conidiate in submerged culture.

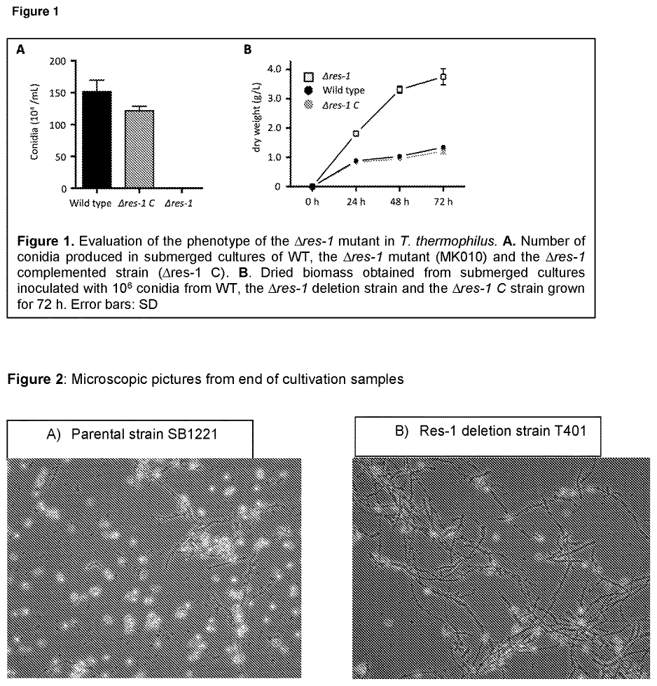

Conidiation and biomass production in liquid culture of wild type, the Δres-1 deletion strain and the complemented Δres-1 (Δres-1 C) strain by the introduction of a res-1-gfp construct were then compared. The Δres-1 variant failed to produce conidia in submerged culture, a phenotype that was complemented by the introduction of a wild type copy of res-1 (FIG. 1A). Significantly, the Δres-1 mutant produced more fungal biomass in culture, while the WT and Δres-1 C strains stalled in biomass production after 24 h in culture (FIG. 1B).

### Example 2—Transformation of T. thermophilus

Protoplasts of T. thermophilus strains were prepared by inoculating a 25 ml preculture of a standard fungal growth media with 0.7-1×10⁵ spores/ml in a 100 ml shake flask for 24 h at 37° C. and 250 rpm. The main culture was prepared by inoculating 100 ml of a standard fungal growth media with 20 ml of the preculture in a 500 ml shake flask for 24 h at 37° C. and 250 rpm. The mycelium was harvested by filtration through a sterile Cell Strainer (VWR) and washed with 100 ml 2000 mosmol/L NaCl/CaCl₂ (0.6 M NaCl and 0.27 M CaCl₂*H₂O). 1 g of the washed mycelium was transferred into a 100 ml flask. The mycelium was mixed with 150 mg VinoTaste Pro solution (2.5 mg/ml in 2000 mosmol/L NaCl/CaCl2) and 10 mg Yatalase solution (0.625 mg/ml in 2000 mosmol/L NaCl/CaCl₂) and 10 ml of 2000 mosmol/L NaCl/CaCl₂. The mycelium suspension was incubated at 30° C. and 70 rpm for 50-70 min until protoplasts are visible under the microscope. Harvesting of protoplasts was done by filtration through a sterile Cell Strainer into a sterile 50 ml tube. After the addition of 25 ml ice-cold STC solution (1.2 M sorbitol, 50 mM CaCl2, 35 mM NaCl, 10 mM Tris/HCl pH 7.5) to the flow through, the protoplasts were harvested by centrifugation (1200×g, 10 min, 4° C.). The protoplasts were washed again in 50 ml STC and resuspended in 0.5-1.2 ml STC.

For transformation, 5-10 μg of linearized DNA, 1 μl 0.5 M aurintricarboxylic acid (ATA) and 100 μl of protoplast suspension were mixed and incubated for 30-40 min on ice. Then 1.7 ml of PEG solution (60% PEG4000 [polyethylenglycol], 50 mM CaCl2, 35 mM NaCl, 10 mM Tris/HCl pH 7.5) was added and mixed gently. After incubation for 30 min at 4° C., the tube was filled with 11 ml STC solution, centrifuged (900×g, 10 min, 4° C.), and the supernatant discarded. The pellet was resuspended in the remaining STC and plated on selective media plates as known in the art. After incubation of the plates for 3-6 days at 37° C., transformants were picked and re-streaked on selective media.

Selective media plates: If the amdS gene is used as selection marker, enriched minimal medium with uridine and uracil and with acetamide was used to select positive transformants (sucrose is only added in case protoplasts are plated):

| | | |
|---|---|---|
| Glucose | 10 | g/L |
| Sucrose | 230 | g/L |
| Mg₂SO₄*7H₂O | 0.49 | g/L |
| KCl | 0.52 | g/L |
| KH₂PO₄ | 1.52 | g/L |
| CuSO₄*5H₂O | 1.6 | mg/L |
| FeSO₄*7H₂O | 5 | mg/L |
| ZnSO₄*7H₂O | 22 | mg/L |
| MnSO₄*H₂O | 4.3 | mg/L |
| CoCl₂*6H₂O | 1.6 | mg/L |
| Na₂MoO₄*2H₂O | 1.5 | mg/L |
| H₃BO₃ | 11 | mg/L |
| CsCl | 2.52 | g/L |
| EDTA | 50 | mg/L |
| Penicillin | 50000 | U/L |
| Streptomycin | 50 | mg/L |
| Uracil | 1.12 | g/L |
| Uridine | 2.44 | g/L |
| Acetamide | 0.6 | g/L |
| Agar | 16 | g/L |

set pH to 6.5

If the pyr5 gene is used as selection marker, enriched minimal medium without uridine and uracil was used to select positive transformants (sucrose is only added in case protoplasts are plated):

| | | |
|---|---|---|
| Glucose | 10 | g/L |
| Sucrose | 230 | g/L |
| Mg₂SO₄*7H₂O | 0.49 | g/L |
| KCl | 0.52 | g/L |
| KH₂PO₄ | 1.52 | g/L |
| NaNO₃ | 6 | g/L |
| CuSO₄*5H₂O | 1.6 | mg/L |
| FeSO₄*7H₂O | 5 | mg/L |
| ZnSO₄*7H₂O | 22 | mg/L |
| MnSO₄*H₂O | 4.3 | mg/L |
| CoCl₂*6H₂O | 1.6 | mg/L |
| Na₂MoO₄*2H₂O | 1.5 | mg/L |
| H₃BO₃ | 11 | mg/L |
| EDTA | 50 | mg/L |
| Penicillin | 50000 | U/L |
| Streptomycin | 50 | mg/L |
| Casaminoacids | 1 | g/L |
| Agar | 16 | g/L |

set pH to 6.5

If the nat1 gene is used as additional selection marker, enriched minimal medium without uridine and uracil and with nourseothricin (clonNAT) was used to select positive transformants (sucrose is only added in case protoplasts are plated):

| | | |
|---|---|---|
| Glucose | 10 | g/L |
| Sucrose | 230 | g/L |
| Mg₂SO₄*7H₂O | 0.49 | g/L |
| KCl | 0.52 | g/L |
| KH₂PO₄ | 1.52 | g/L |
| NaNO₃ | 6 | g/L |
| CuSO₄*5H₂O | 1.6 | mg/L |
| FeSO₄*7H₂O | 5 | mg/L |
| ZnSO₄*7H₂O | 22 | mg/L |
| MnSO₄*H₂O | 4.3 | mg/L |
| CoCl₂*6H₂O | 1.6 | mg/L |
| Na₂MoO₄*2H₂O | 1.5 | mg/L |
| H₃BO₃ | 11 | mg/L |
| EDTA | 50 | mg/L |
| Penicillin | 50000 | U/L |
| Streptomycin | 50 | mg/L |
| Nourseothricin | 35 | mg/L |
| Casaminoacids | 1 | g/L |
| Agar | 16 | g/L |

set pH to 6.5

### Example 3—Phytase Activity Assay

The phytase activity was determined in microtiter plates. The phytase containing supernatant was diluted in reaction buffer (250 mM Na-acetate, 1 mM CaCl₂, 0.01% Tween 20, pH 5.5). 10 μl of the phytase enzyme solution was incubated with 140 μl substrate solution (6 mM Na-phytate (Sigma P3168) in reaction buffer) for 1 h at 37° C. The reaction was quenched by adding 150 μl of trichloroacetic acid solution (15% w/w). To detect the liberated phosphate, 20 μl of the quenched reaction solution was treated with 280 μl of freshly made-up color reagent (60 mM L-ascorbic acid (Sigma A7506), 2.2 mM ammonium molybdate tetrahydrate, 325 mM H₂SO₄), and incubated for 20 min at 37° C., and the absorption at 820 nm was subsequently determined. For the blank value, the substrate buffer on its own was incubated at 37° C. and the 10 μl of enzyme sample was only added after quenching with trichloroacetic acid. The color reaction was performed analogously to the remaining measurements. The amount of liberated phosphate was determined via a calibration curve of the color reaction with a phosphate solution of known concentration.

### Example 4—Generation of Plasmids for Transformation of T. thermophilus

Phytase expression plasmid for integration at cbh1 locus: A synthetic gene (GeneArt, ThermoFisher Scientific Inc., USA) encoding a synthetic phytase from bacterial origin (disclosed in WO 2012/143862; as phytase PhV-99; SEQ ID NO: 1) was used for the construction of a phytase expression plasmids. For the secretion of the phytase, a signal sequence encoding for a signal peptide derived from T. thermophilus was added to the mature sequence of the phytase. A promotor sequence amplified from the upstream region of the TEF1 (XP_003660173.1) encoding gene and a terminator sequence amplified from the downstream region of the Cbh1 (XP_003660789.1) encoding gene from T. thermophilus were used as regulatory elements to drive the expression of the phytase. Using standard cloning techniques, the expression plasmid MT2703 (SEQ ID NO: 2) was constructed based on the E. coli cloning plasmid MT940 (SEQ ID NO: 3). Plasmid MT940 consists of the pMB1 origin of replication, kan resistance, pyr5 gene from T. thermophilus, upstream and downstream regions of the Cbh1 encoding gene from T. thermophilus for homologous recombination, and lacZ for blue/white screening. The expression plasmid MT2703 contains the upstream region of cbh1 from bases 1913-3345, the TEF1 promotor sequence from bases 3350-5847, the phytase including a signal sequence from bases 5850-7163, the cbh1 terminator sequence from bases 7168-7374, and the downstream region of cbh1 from bases 8666-10295.

The plasmid was digested with NotI to remove the vector backbone and the fragment containing the phytase expression cassette was isolated from an agarose gel. Only the isolated DNA fragment was later used for transformation.

Deletion constructs for res-1 locus: For the deletion of the res-1 (XP_003662015.1, SEQ ID NO: 4) locus a split-marker approach was used. Therefore two deletion plasmids were cloned, each carrying a non-functional part of a nourseothricin acetyltransferase gene (nat1) selection marker cassette. Both fragments share a 610 bp overlap, which allows the in vivo homologous recombination of the two marker fragments thereby restoring the functional full length marker cassette.

Using standard PCR and cloning methods, an approx. 1.5 kb fragment of the upstream region of the Res-1 encoding gene from T. thermophilus was fused to the 5′-fragment of the split nat1 selection marker cassette and cloned into the E. coli cloning vector MT944 (SEQ ID NO: 5). Plasmid MT944 consists of the pUC origin of replication, kan resistance, and lacZ for blue/white screening. This results in the deletion plasmid MT2013 (SEQ ID NO: 6). MT2013 contains the upstream region of res-1 from bases 266-1822 and the partial nat1 selection marker cassette from bases 1841-2687. An approx. 1.5 kb fragment of the downstream region of the Res-1 encoding gene was fused to the 3′-fragment of the split nat1 selection marker cassette and cloned into MT944, resulting in the deletion plasmid MT2014 (SEQ ID NO: 7). MT2014 contains the partial nat1 selection marker cassette from bases 1671-2448 and the downstream region of res-1 from bases 2462-3995.

The res-1 deletion plasmids MT2013 and MT2014 were digested with NotI to remove the vector backbone and the fragments containing the split nat1 selection marker cassettes were isolated from an agarose gel. Only the isolated DNA fragments were later used for transformation.

### Example 5—Generation T. thermophilus Strains with Phytase Expression Construct Integrated at cbh1 Locus

For the expression of a heterologous phytase, the T. thermophilus host strain UV18 #100f Δpyr5 Δalp1 Δku70 (construction described in detail in International Publication No. WO 2017/093450, the disclosure of which is incorporated herein by reference) from the C1 lineage, a strain with uracil auxotrophy, reduced protease activity, and impaired non-homologous end joining (NHEJ) repair system, was transformed as described in Example 1 with the NotI-digested and isolated phytase expression constructs (as described in Example 3) from plasmid MT2703 (SEQ ID NO: 2). The transformants were incubated for 3-6 days at 37° C. on enriched minimal medium for pyr5 selection to select for restored uracil prototrophy by complementing the pyr5 deletion with the pyr5 marker as known in the art. Colonies were re-streaked and checked for the integration of the phytase expression cassette using PCR with primer pairs specific for the phytase expression cassette and the cbh1 locus as known in the art. A transformant tested positive for the phytase expression construct at the cbh1 locus was named SB1221 selected for further characterization.

### Example 6—Deletion of the res-1 Gene in the Phytase Expression Strain SB1221

The phytase production strain SB1221 was transformed as described in Example 1 with the two NotI-digested and isolated res-1 deletion cassettes containing the split nat1 selection marker cassettes (as described in Example 3) from plasmid MT2013 and MT2014. The transformants were incubated for 3-6 days at 37° C. on enriched minimal medium for nat1 selection to select for resistance to nourseothricin (clonNAT). Colonies were re-streaked and checked for the integration of the recombined nat1 selection marker and the deletion of the res-1 locus using PCR with primer pairs specific for nat1 and the res-1 locus. A transformant tested positive for the deletion of the res-1 locus was named T401 selected for further characterization.

### Example 7—Enzyme Production in Small Scale Cultivation with res-1 Deletion Strain T401

Generated res-1 mutant strain T401 together with the parental strain SB1221 were fermented in small-scale cultivation and the supernatants were analyzed. T. thermophilus strains grown on agar were inoculated in 1 ml cultivation medium as shown below in Table 1 in a 96-deepwell microtiter plate. The strains were fermented at 37° C. on a microtiter plate shaker at 900 rpm and 80% humidity for 72 hours. 300 μl of the 72-hour-preculture were transferred in 700 μl fresh cultivation medium in a 96-deepwell microtiter plate. The strains were fermented at 37° C. on a microtiter plate shaker at 900 rpm and 80% humidity for 96 hours. All strains were cultivated in hexaplicate.

TABLE 1
Cultivation medium

| Ingredient | Amount | |
|---|---|---|
| Glucose | 10 | g/L |
| Mg₂SO₄*7H₂O | 0.49 | g/L |
| K₂SO₄ | 1.21 | g/L |
| CaSO₄*2H₂O | 0.47 | g/L |
| KH₂PO₄ | 1.75 | g/L |
| (NH₄)₂SO₄ | 4.63 | g/L |
| CuSO₄*5H₂O | 1.46 | mg/L |
| FeSO₄*7H₂O | 4.56 | mg/L |
| ZnSO₄*7H₂O | 20.05 | mg/L |
| MnSO₄*H₂O | 3.92 | mg/L |
| Na₂MoO₄*2H₂O | 1.37 | mg/L |
| EDTA | 50 | mg/L |
| Biotin | 0.006 | mg/L |
| Penicillin | 50000 | U/L |
| Streptomycin | 50 | mg/L |
| Casamino acids | 1 | g/L |
| MES | 21.33 | g/L |

set pH to 6.0

At the end of the cultivation, the broth was examined with a microscope. Compared to the culture of the control strain SB1221, which contains mainly spores and only little amounts of mycelium (FIG. 2A), the res-1 deletion strain T401 showed only a few spores and mainly vegetative growing mycelium (FIG. 2B). The deletion of res-1 significantly reduced the conidiation in submers cultivation.

Cell-free supernatants were harvested at the end of cultivation and subjected to a phytase activity assay as described above in Example 2. Deletion of res-1 results in a higher activity of the produced heterologous phytase (Table 2).

TABLE 2
Relative phytase activity of averaged
hexaplicate cultivations in a microtiter plate.

| strain | phytase activity |
|---|---|
| SB1221 | 100% |
| T401 | 110% |

### Example 8—Enzyme Production with a res-1 Deletion Mutant of T. thermophilus in a Stirred Tank Reactor

Pre-cultures of T. thermophilus were prepared by inoculation of 175 mL of pre-culture medium with 104 spores/mL in a 1 L shaking flask and incubated for 72 h at 35° C. and 250 rpm. Alternatively, pre-cultures can be inoculated by frozen mycelial stocks of T. thermophilus without any influence on process performance or protein yields. For detailed pre-culture media composition, see Tables 3 and 4 below.

TABLE 3
pre-culture medium

| | Concentration |
|---|---|
| Component | [g/kg] |
| Glucose × H₂O | 8.80 |
| (NH₄)₂SO₄ | 4.66 |
| MgSO₄ × 7 H₂O | 0.49 |
| KCl | 0.52 |
| CaCl₂ × 2 H₂O | 0.40 |
| KH₂PO₄ | 10.2 |
| Biotin stock solution (6 mg/L) | 1.0 |
| Casaminoacids | 1.0 |
| Pen/Strep solution (2 g/L Penicillin G/5 g/L Streptomycin) | 1.0 |
| Trace element solution | 1.0 |

TABLE 4
Trace element solution

| Component | Concentration [g/kg] |
|---|---|
| EDTA | 50.0 |
| ZnSO₄ × 7 H₂O | 20.05 |
| H₃BO₃ | 10.03 |
| MnSO₄ × H₂O | 3.92 |
| FeSO₄ × 7 H₂O | 4.56 |
| CoCl₂ × 6 H₂O | 1.55 |
| CuSO₄ × 5 H₂O | 1.46 |
| Na₂MoO₄ × 2H₂O | 1.37 |

Extended fed-batch cultivations were carried out in an Ambr®250 Modular bench top bioreactor system (Sartorius). The pre-cultures were aseptically transferred to the stirred tank reactor. The inoculum volume used was 8% of the starting volume of 180 mL. The media composition used for fed-batch cultivation is given in Table 5.

TABLE 5
Main culture medium.

| | Concentration |
|---|---|
| Component | [g/kg] |
| (NH₄)₂SO₄ | 10.1 |
| MgSO₄ × 7 H₂O | 0.53 |
| CaCl₂ × 2 H₂O | 0.43 |
| KH₂PO₄ | 1.64 |
| KCl | 0.56 |
| Glucose × H₂O | 17.6 |
| Trace element solution | 1.0 |
| Biotin stock solution (6 mg/L) | 1.0 |
| Pen/Strep solution (2 g/L Penicillin G/5 g/L Streptomycin) | 1.0 |
| Antifoam Adekanol LG109 | 1.0 |

Cultivations were performed at a temperature of 37° C., initial stirrer speed of 1500 rpm, gassing with air 0.18 L/min. DOT (Dissolved oxygen tension) was controlled at >20% by adjusting the stirrer speed. The pH was set to pH 6.0 and was controlled using 15% NH₄OH solution. Feeding of 50% (w/w) glucose solution started at the end of the of batch phase when the pH increased up to pH=6.3, which happened 16-20 h after inoculation. The feeding rate was set to 0.25 mL/h which resulted in a glucose limitation during the feed phase of the fermentation. The fermentation runs for 70 h.

Broth samples were withdrawn throughout the fermentation. Cell free supernatant was obtained by filtration of the broth with 0.22 μm filters and was used to analyze protein concentrations and phytase activities. Protein concentrations were determined using a Cedex Bio Analyzer (Roche CustomBiotech). Phytase activities were determined as described above.

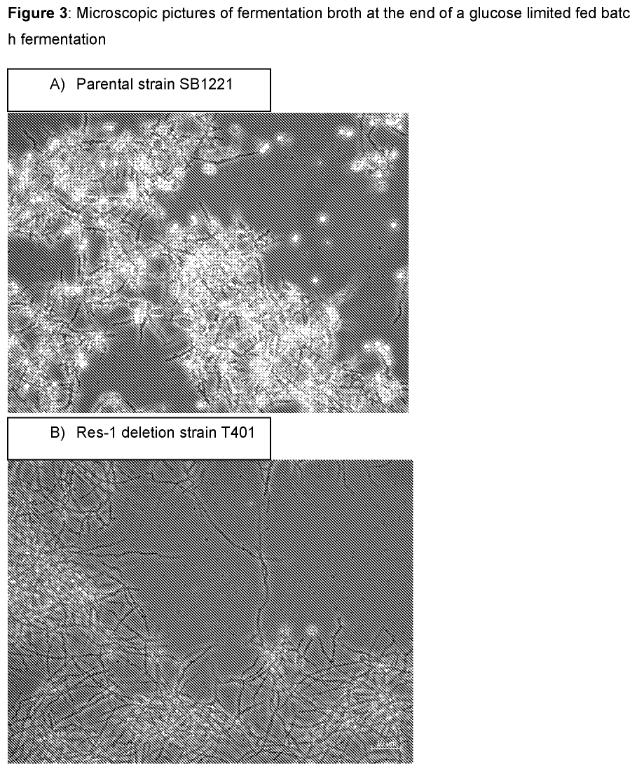

A microscopic analysis of the broth samples showed a lot of spores for parental strain SB1221 (FIG. 3A), while no spores could be detected for res-1 deletion strain T401 (FIG. 3B).

Deletion of res-1 in strain T401 provides fermentation broth with higher secreted protein concentration and of higher phytase activity in the supernatant compared to the parental strain SB1221. See Table 6.

TABLE 6
Relative total protein concentration and phytase activity in the
supernatant at the end of a carbon limited fed batch fermentation.

| Strain | total protein | phytase activity |
|---|---|---|
| SB1221 | 100% | 100% |
| T401 | 200% | 111% |

1. A method of reducing conidiation during cell culture of a T. thermophilus strain, the method comprising growing, in a submerged cell culture, a T. thermophilus strain that has reduced res-1 activity as compared to a wild-type T. thermophilus strain.

2. The method of claim 1, wherein the variant T. thermophilus strain comprises a genetic modification in a res-1 gene that results in reduced res-1 activity.

3. The method of claim 2, wherein the modification is a knock-out mutation in the res-1 gene.

4. The method of claim 1, wherein the T. thermophilus strain further comprises a nucleic acid encoding a heterologous protein.

5. The method of claim 4, wherein the nucleic acid encoding the heterologous protein is operably linked to a promoter.

6. The method of claim 1, wherein the variant T. thermophilus strain is grown using a fed-batch fermentation process.

7. The method of claim 1, wherein the variant T. thermophilus strain is grown in a fermenter.

8. The method of claim 1, wherein the variant T. thermophilus strain is grown under carbon-limited conditions.

9. The method of claim 1, wherein the variant T. thermophilus strain is grown under glucose-limited conditions.

10. A method of increasing production of a heterologous protein in a T. thermophilus strain, the method comprising growing a T. thermophilus strain comprising a nucleic acid encoding a heterologous protein in a submerged culture, wherein the T. thermophilus strain has reduced res-1 activity, wherein the expression level of the heterologous protein produced by the T. thermophilus strain that has reduced res-1 activity is higher than an expression level of a recombinant protein produced by a T. thermophilus strain having wild type res1 activity.

11. The method of claim 10, wherein the variant T. thermophilus strain comprises a genetic modification in a res-1 gene that results in reduced res-1 activity.

12. The method of claim 11, wherein the modification is a knock-out mutation in the res-1 gene.

13. The method of claim 4, wherein the heterologous protein is an enzyme.

14. The method of claim 13, wherein the enzyme is phytase, hydrolase, isomerase, ligase, lyase, oxidoreductase, transferase, aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, nuclease, oxidase, pectinolytic enzyme, peroxidase, phosphodiesterase, polyphenoloxidase, proteolytic anzyme, ribonuclease, transglutaminase, xylanase, and beta-xylosidase.

15. The method of claim 13, wherein the heterologous protein is phytase.
